(19) United States
(12) Patent Application Publication
(10) Pub. No.: US 2020/0318189 A1
(43) Pub. Date: Oct. 8, 2020

(54) METHOD FOR ASSESSING THE RISK OF CUTANEOUS ADVERSE DRUG REACTIONS INDUCED BY ANTI-EPILEPTIC DRUG LAMOTRIGINE, DETECTION REAGENT THEREOF AND USE THEREOF

(71) Applicants: Shuen-Iu HUNG, Taoyuan County (TW); CHANG GUNG MEMORIAL HOSPITAL, LINKOU, Taoyuan City (TW)

(72) Inventors: Shuen-Iu HUNG, Taoyuan County (TW); Wen-Hung CHUNG, Taoyuan County (TW)

(21) Appl. No.: 16/759,151

(22) PCT Filed: Oct. 27, 2017
(86) PCT No.: PCT/CN2017/107933

§ 371 Date: Apr. 24, 2020

(51) Int. Cl. C12Q 1/6883; C12Q 1/6881

(52) U.S. Cl. CPC C12Q 1/6883; C12Q 2600/106; C12Q 2600/156; C12Q 1/6881

(57) ABSTRACT

A method for assessing the risk of cutaneous adverse drug reactions induced by an anti-epileptic drug Lamotrigine is provided, wherein the cutaneous adverse drug reactions include but are not limited to: maculopapular eruption (MPE), fixed drug eruption (FDE), Stevens Johnson Syndrome (SJS), toxic epidermal necrolysis (TEN), drug rash with eosinophilia and systemic symptoms (DRESS). Further provided is a detection reagent for assessing the risk of a patient developing the cutaneous adverse drug reactions induced by the anti-epileptic drug Lamotrigine, comprising agents for measuring a particular HLA allele and the use thereof.

### TECHNICAL FIELD

The present invention provides a method for assessing the risk of developing a cutaneous adverse drug reaction, and more particular, to a method for assessing the risk of developing a cutaneous adverse reaction induced by the anti-epileptic drug Lamotrigine.

### BACKGROUND TECHNIQUE

Cutaneous Adverse Drug Reactions (CADRs) have always been a major clinical problem with very diverse manifestations, ranging from maculopapular eruption (MPE), fixed drug eruption (FDE) to severe cutaneous adverse drug reactions (SCARs), which includes drug rash with eosinophilia and systemic symptoms (DRESS), Stevens Johnson Syndrome (Stevens Johnson Syndrome, SJS) and toxic epidermal necrolysis (TEN). MPE is the most common drug eruption, usually begins with scattered small red spots and papules and then merge into a diffuse rash. Fixed drug eruption (FDE) refers to a drug rash that erupts in the same location after the patient taking the same drug each time. The most common locations are the face, lips, external genitalia and extremities. Signs usually are round or oval reddish-purple plaques associated with itching and stinging. The plaques can be single or multiple and can become large blisters in more severe cases. After the acute phase, brownish black pigmentation will remain. Stevenson Johnson Syndrome (SJS) and Toxic Epidermal Necrosis (TEN) often have prodromal symptoms similar to that of the common cold, including fever, sore throat, swelling of the lips, etc., followed by rapid eruption of systemic erythema, blisters and inflamed and ulcerated mucosa in the eyes, mouth and genital. In severe cases, the patients appear to have whole body burn. The biggest difference between SJS and TEN is that in the former, the epidermal separation is less than 10% of the body surface area, and TEN has more than 30% of epidermal separation. The main clinical features of drug eruption with eosinophilia and systemic symptoms (DRESS) include fever, skin rash, increased eosinophils in the blood, enlarged lymph nodes, and internal organ involvement. The most common and serious organ involvement is the liver, which may lead to fulminant hepatitis, the most common cause of death in these patients. Other organ involvement include nephritis, myocarditis, pneumonia, and thyroiditis.

Adverse drug reactions are often associated with immune responses. If a patient is allergic to a specific drug and consumed the same drug, the second adverse drug reaction develops more rapidly and is more serious. However, the immune mechanism is very complicated, for example, HLA-A has more than 300 genotypes; HLA-B has more than 600 genotypes. Therefore, it is difficult to find the immune mechanism that underlines the adverse drug reactions.

Lamotrigine (LTG) (trade name: Lmital™, hereinafter referred to as LTG) is an anti-epileptic drug currently available in the clinical setting. Its mechanism of action is by inhibiting voltage sensitive sodium ion channel, which in turn reduces the release of glutamate at the synaptic terminal and achieves the effect of nerve stabilization. Compared with traditional anti-epileptic drugs, LTG is a relatively new drug developed in the late 1980s. Its structure is slightly different from that of the traditional anti-epileptic drugs and belongs to the triazine group. According to the directive of the US Food and Drug Administration (FDA), LTG is currently approved for the treatment of focal seizures, generalized seizures, Lennox-Gastaut syndrome and type 1 bipolar disorder, while other off-label uses include weight loss, migraine, trigeminal neuralgia, and depression [Micomedex].

Although LTG can be used to treat many types of epilepsy, its use is limited due to a higher incidence of adverse reactions in the clinical setting. The most serious side effects in the clinical setting are severe cutaneous adverse reactions, including: SJS, TEN and DRESS. DRESS also causes systemic reactions such as liver failure, anemia, the reduction of granular cells, thrombocytopenia, peripheral intravascular agglutination and eosinophilia. Therefore, there is still a need for assessing the risk of LTG-induced cutaneous adverse drug reaction. The present invention addresses this need.

### SUMMARY OF THE INVENTION

The present invention provides a method for assessing the risk of developing a cutaneous adverse drug reaction induced by an anti-epileptic drug, Lamotrigine, wherein the cutaneous adverse drug reaction comprises: maculopapular eruption (MPE), fixed drug eruption (FDE) and severe cutaneous adverse drug reactions (SCAR), wherein said SCAR comprises Stevens Johnson Syndrome (SJS), toxic epidermal necrosis (TEN) or drug rash with eosinophilia and systemic symptoms (DRESS). The present invention is directed to the discovery of the association of HLA-A*0207 and/or HLA-B*1502 allele and cutaneous adverse drug reactions caused by the anti-epileptic drug, lamotrigine.

In particular, the present invention provides a method for assessing the risk of developing a cutaneous adverse drug reaction in a patient, comprising the step of detecting the presence of an HLA-A*0207 and/or HLA-B*1502 allele, wherein the wherein the presence of HLA-A*0207 and/or HLA-B*1502 allele is an indicator of the risk of cutaneous adverse drug reaction. In one embodiment, the drug is the anti-epileptic drug Lamotrigine. Cutaneous adverse drug reaction includes at least one reaction selected from the group consisting of maculopapular eruption (MPE), fixed drug eruption (FDE), and Stevens Johnson Syndrome (SJS), toxic epidermal necrosis (TEN) or drug rash accompanied by eosinophilia and systemic symptoms (DRESS). In one embodiment, the patient carries the HLA-A*0207 allele. In one embodiment, the patient carries HLA-B*1502. In another embodiment, the patient carries HLA-A*0207 and HLA-B*1502.

In one embodiment, the patient carries HLA-A*0207 and the cutaneous adverse drug reaction is Stevens Jonson Syndrome, toxic epidermal necrosis or drug eruption with eosinophilia and systemic symptoms. In one embodiment, the patient carries HLA-A*0207 and the cutaneous adverse drug reaction is a maculopapular rash or a fixed drug eruption. In one embodiment, the patient carries HLA-B*1502 and the cutaneous adverse drug reaction is maculopapular rash or fixed drug eruption. In one embodiment, the patient has HLA-B*1502, and the cutaneous adverse drug reaction is maculopapular rash, fixed drug eruption or drug eruption accompanied by eosinophilia and systemic symptoms. In one specific embodiment, the patient has HLA-A*0207 and HLA-B*1502, and the cutaneous adverse drug reaction is Stevens Jonson syndrome, toxic epidermal necrosis or drug eruption with eosinophilia and systemic symptom. In one specific embodiment, the patient has HLA-A*0207 and HLA-B*1502, and the cutaneous adverse drug reaction is a maculopapular rash or a fixed drug eruption. In another specific embodiment, the patient carries HLA-A*0207 or HLA-A*0207 plus HLA-B*1502, and the cutaneous adverse drug reaction includes at least one adverse reaction selected from the group consisting of maculopapular rash, fixed drug eruption, Stevens Johnson Syndrome, toxic epidermal necrosis or drug eruption with eosinophilia and systemic symptoms.

The present invention provides an agent for detecting the HLA-A*0207 and/or HLA-B*1502 alleles in the manufacture of a detection reagent to evaluate the risk of developing a cutaneous adverse drug reaction induced by an anti-epileptic drug Lamotrigine. The agent detects the presence of the HLA-A*0207 and/or HLA-B*1502 allele. The cutaneous adverse drug reaction includes at least one adverse reaction selected from the group consisting of maculopapular rash, fixed drug eruption, Stevens Johnson Syndrome, toxic epidermal necrosis or drug eruption accompanied by eosinophilia and systemic symptoms.

The presence of HLA-A*0207, HLA-B*1502 or HLA-A*0207 and HLA-B*1502 alleles indicates the patient is higher than one time, higher than two times, higher than three times, higher than four times, higher than five times, higher than six times, higher than seven times, higher than eight times, higher than nine times, higher than ten times, higher than three times to four times, or higher than one time to four times risk of developing cutaneous adverse drug reaction compared to a subject without the HLA-A*0207, HLA-B*1502 or HLA-A*0207 and HLA-B*1502 alleles.

Methods known in the art for detecting alleles can be used, such as (but not limited to): an oligonucleotide that specifically hybridizes to the allele, serotyping or microcytotoxicity method to determine cDNA, RNA or protein product of the allele. [Kenneth D. McClatchey.Clinical Laboratory Medicine.2002]. In one embodiment, the oligonucleotide specifically hybridizes to the DNA of the peripheral blood of the patient. The oligonucleotide is designed for the most variable sequences of HLA-A*0207 and/or HLA-B*1502 alleles. For example, as shown in FIG. 1, exon 2 (SEQ ID NO:2) and exon 3 (SEQ ID NO:1) of the HLA-A*0207 and HLA-B*1502 alleles.

In one exemplary embodiment, oligonucleotide sequence of the forward primer used to detect the presence of HLA-A*0207 is 5′-TCCGGAGTATTGGGACGGGGAGACACGGAAA-3′ (SEQ ID NO. 3), sequence of the reverse primer is 5′-TCAACTGCTCCGCCACATGGGCCGCCT-3′ (SEQ ID NO: 4) and the sequences of the probes are 5′-TCCAGAGGATGTGTGGCT-3′ (SEQ ID NO: 5) and 5′-TCCAGAGGATGTATGGCT-3′ (SEQ ID NO: 6). In another exemplary embodiment, oligonucleotide sequence of the forward primer used to detect the presence of HLA-B*1502 is 5′-ATGGCGCCCCGGG-3′ (SEQ ID NO:7), the sequence of the reverse primer is 5′-TAGTAGCCGCGCAGGTTCC-3′ (SEQ ID NO: 8), the probes sequences are 5′-AACACACAGATCTACAAGG-3′ (SEQ ID NO: 9) and 5′-AACACACAGATCTCCAAGA-3′ (SEQ ID NO: 10). In another embodiment, the serotyping method or microcytotoxicity method is performed using RNA, protein, cells or serum from the peripheral blood of the patient.

The present invention provides a reagent for evaluating the risk of developing a cutaneous adverse drug reaction induced by an anti-epileptic drug Lamotrigine, by detecting the presence of HLA-A*0207 and/or HLA-B*1502 alleles, wherein the presence of HLA-A*0207 and/or HLA-B*1502 alleles indicates the risk of developing a cutaneous adverse drug reaction, and said cutaneous adverse drug reaction includes at least one adverse reaction selected from the group consisting of maculopapular rash, fixed drug eruption, Stevens Johnson Syndrome, toxic epidermal necrosis or drug eruption accompanied by eosinophilia and systemic symptoms.

The terms “invention” and “present invention” as used in the present invention are intended to broadly refer to the application the claims. The statements containing these terms are to be understood as not limiting the scope of the application or the scope of the claims. The working examples of the invention are defined by the application and not by the content of the present invention. This summary is a high-level overview of various aspects of the invention and is a description of some concepts that are further described in the section below. This Summary is not intended to identify key or essential features of the claimed application, and is not intended to be used solely to determine the scope of the claimed application. The objectives of the application should be understood by reference to any or all of the figures and the appropriate parts of each claim.

### FIGURES

FIG. 1 shows the exon sequences (exon2 and exon3) of the HLA-A*0207 and HLA-B*1502 alleles. The lowercase sequence is an intron connecting exon2 and exon3.

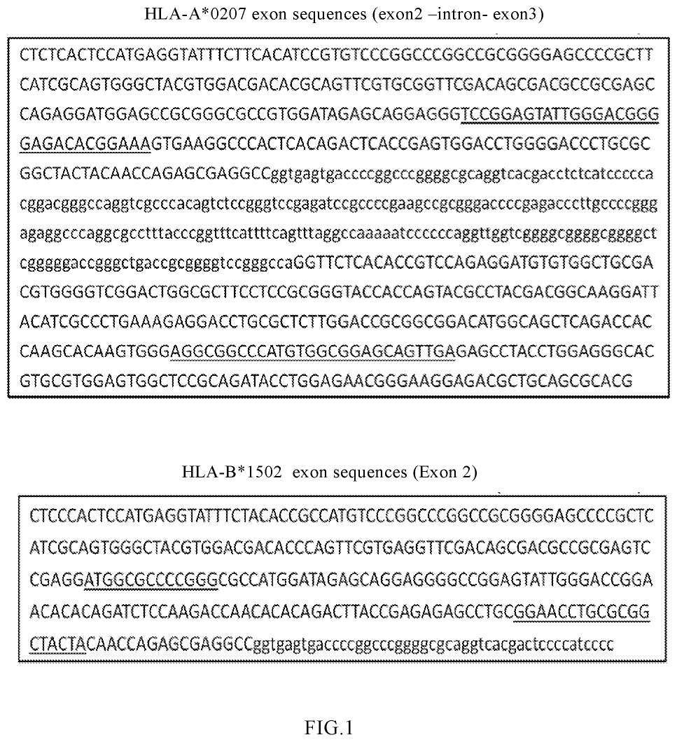

### WORKING EXAMPLE

The present invention provides a method for assessing the risk of developing a cutaneous adverse drug reaction induced by an anti-epileptic drug, Lamotrigine, wherein said cutaneous adverse drug reaction comprises at least one adverse reaction selected from the group consisting of maculopapular eruption (MPE) a fixed drug eruption (FDE) and severe cutaneous adverse drug reactions (SCAR), and said SCAR comprises at least one severe cutaneous adverse drug reaction selected from the group consisting of: Stevens Johnson Syndrome (SJS), toxic epidermal necrolysis (TEN) or drug rash with eosinophilia and systemic symptoms (DRESS). The present invention is directed to the discovery that HLA alleles, HLA-A*0207, HLA-B*1502 or HLA-A*0207 and HLA-B*1502 are associated with the cutaneous adverse drug reaction of induced by the anti-epileptic drug lamotrigine.

In the following examples, 26 patients with lamotrigine induced cutaneous adverse drug reaction (including SCAR, MPE, and FDE patients) were enrolled for HLA typing and compared with 755 normal healthy controls. The results show that HLA-A*0207, HLA-B*1502 or HLA-A*0207 and HLA-B*1502 alleles were associated with lamotrigine-induced cutaneous adverse drug reaction (Table 1).

With respect to the HLA-A*0207 allele, 11 of the 26 patients with lamotrigine induced cutaneous adverse drug reaction (CADR) carried this genotype (42.31%) whereas only 200 of the 755 normal healthy controls (Control) carried this genotype (16.82%). This shows the association of HLA-A*0207 with lamotrigine induced CADR (CADR vs. Control: P=2.48×10⁻³, Odds Ratio (OR)=3.63 (1.63-8.08), sensitivity: 42.31%, specificity: 83.18%). With respect to the HLA-B*1502 allele, 7 of the 26 patients with lamotrigine induced cutaneous adverse drug reaction—(CADR) carried this genotype (26.92%), and only 73 of the 755 normal healthy controls carried this genotype (10.70%). This shows the association of HLA-B*1502 with lamotrigine induced CADR (CADR vs. Controls): P=1.2×10⁻², odds ratio=3.44 (1.40-8.46), sensitivity: 26.92%, specificity: 90.33%). Further analysis of HLA-A*0207 and HLA-B*1502 alleles showed the sensitivity for assessing the risk of lamotrigine induced CADR significantly increased (CADR vs. Controls: P=4.32×10⁻⁴, odds ratio=4.44 (1.98-9.45), sensitivity: 61.54%, specificity: 73.51%). The above results show that the presence or absence of HLA-A*0207, HLA-B*1502 or HLA-A*0207 and HLA-B*1502 alleles can be used to evaluate the risk of developing a cutaneous adverse drug reaction induced by the anti-epileptic drug, lamotrigine.

TABLE I
Analysis and Comparison of the HLA-A*0207 and/or
HLA-B*1502 genotype in 26 patients with lamotrigine
induced CADR and 755 normal healthy controls.

| | LTG-CADR | | Control | | | | | |
|---|---|---|---|---|---|---|---|---|
| | (n = 26) | | (n = 755) | | | OR | Sensitivity | Specificity |
| | (+) | (−) | (+) | (−) | P Value | (95% CI) | (%) | (%) |
| HLA-A*0207 | 11 | 15 | 127 | 628 | 2.48 × 10⁻³ | 3.63 | 42.31 | 83.18 |
| | | | | | | (1.63-8.08) | | |
| HLA-B*1502 | 7 | 19 | 73 | 682 | 1.20 × 10⁻² | 3.44 | 26.92 | 90.33 |
| | | | | | | (1.40-8.46) | | |
| HLA-A*0207 | 16 | 10 | 200 | 555 | 4.32 × 10⁻⁴ | 4.44 | 61.54 | 73.51 |
| and | | | | | | (1.98-9.95) | | |
| HLA-B*1502 | | | | | | | | |

Accordingly, the present invention provides a method for assessing the risk of a developing a cutaneous adverse drug reaction of a patient after taking a drug, comprising the step of determining the presence of an HLA-A*0207 and/or HLA-B*1502 allele, wherein the presence of HLA-A*0207 and/or HLA-B*1502 alleles is an indicator of the risk of developing the cutaneous adverse drug reaction. The drug is the anti-epileptic drug Lamotrigine. The cutaneous adverse drug reaction includes at least one adverse reaction selected from the group consisting of maculopapular rash (MPE), fixed drug eruption (FDE), Stevens Johnson Syndrome (SJS), toxic epidermal necrosis (TEN), or drug eruption with eosinophilia and systemic symptoms (DRESS).

The foregoing is a description of the preferred embodiments of the present invention, and the present invention will be described in detail, and the subject matter of the present invention can be changed and modified without departing from the spirit and scope of the invention. Modifications are intended to be included within the scope of the following claims.

1. A method for treating an indication treatable by an anti-epileptic, lamotrigine, comprising the steps of
(a) detecting the presence of HLA-A*0207, HLA-B*1502 or HLA-A*0207 and HLA-B*1502 alleles, wherein the presence of HLA-A*0207, HLA-B*1502 or HLA-A*0207 and HLA-B*1502 alleles indicates the patient has a higher risk of developing the cutaneous adverse drug reaction compared to a patient without the HLA-A*0207, HLA-B*1502 or HLA-A*0207 and HLA-B*1502 alleles; and
(b) based on the detecting the presence of HLA-A*0207, HLA-B*1502 or HLA-A*0207 and HLA-B*1502 alleles, treating the indication of the patient by administering an anti-epileptic other than lamotrigine in order to minimize the risk developing a cutaneous adverse drug reaction in the patient caused by lamotrigine.

2. The method according to claim 1, wherein said cutaneous adverse drug reaction comprises at least one of the following adverse reactions: maculopapular eruption (MPE), fixed drug eruption (FDE), and Stevens Johnsons Syndrome (SJS), toxic epidermal necrolysis (TEN) or drug rash with eosinophilia and systemic symptoms (DRESS).

3. The method according to claim 1, wherein said HLA-A*0207, HLA-B*1502 or HLA-A*0207 and HLA-B*1502 alleles are detected in the DNA, RNA, proteins, cells or serum sample prepared from the peripheral blood of the patient.

4-9. (canceled)


SEQUENCE LISTING (10 sequences)

SEQ ID NO: 1 - DNA, 785 bp, Homo Sapiens
  HLA-A*0207 allele Exon 2-Intron-Exon 3
ctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtgtggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacg

SEQ ID NO: 2 - DNA, 313 bp, Homo Sapiens
  HLA-B*1502 allele (Exon 2)
ctcccactccatgaggtatttctacaccgccatgtcccggcccggccgcggggagccccgctcatcgcagtgggctacgtggacgacacccagttcgtgaggttcgacagcgacgccgcgagtccgaggatggcgccccgggcgccatggatagagcaggaggggccggagtattgggaccggaacacacagatctccaagaccaacacacagacttaccgagagagcctgcggaacctgcgcggctactacaaccagagcgaggccggtgagtgaccccggcccggggcgcaggtcacgactccccatcccc

SEQ ID NO: 3 - DNA, 31 bp, Homo Sapiens
  Forward Primer
tccggagtattgggacggggagacacggaaa

SEQ ID NO: 4 - DNA, 27 bp, Homo Sapiens
  Reverse Primer
tcaactgctccgccacatgggccgcct

SEQ ID NO: 5 - DNA, 18 bp, Homo Sapiens
  Probe
tccagaggatgtgtggct

SEQ ID NO: 6 - DNA, 18 bp, Homo Sapiens
  Probe
tccagaggatgtatggct

SEQ ID NO: 7 - DNA, 13 bp, Homo Sapiens
  Forward Primer
atggcgccccggg

SEQ ID NO: 8 - DNA, 19 bp, Homo Sapiens
  Reverse Primer
tagtagccgcgcaggttcc

SEQ ID NO: 9 - DNA, 19 bp, Homo Sapiens
  Probe
aacacacagatctacaagg

SEQ ID NO: 10 - DNA, 19 bp, Homo Sapiens
  Probe
aacacacagatctccaaga